(19) United States
(12) Patent Application Publication
(10) Pub. No.: US 2019/0345092 A1
(43) Pub. Date: Nov. 14, 2019

(54) PREPARATION OF PHENOLIC (METH)ACRYLATES

(71) Applicant: Rohm and Haas Company, Collegeville, PA (US)

(72) Inventors: James H. Pawlow, Aurora, OH (US); Robert Wilczynski, Yardley, PA (US)

(21) Appl. No.: 16/461,481

(22) PCT Filed: Oct. 24, 2017
(86) PCT No.: PCT/US2017/057975

§ 371 Date: May 16, 2019

Related U.S. Application Data
(60) Provisional application No. 62/425,650, filed on Nov. 23, 2016.

(51) Int. Cl. C07C 67/08

(52) U.S. Cl. CPC C07C 67/08; C07C 69/017

(57) ABSTRACT

A method for preparation of phenolic (meth)acrylates. The method comprises contacting contacting acetic anyhydride, a phenolic compound and (meth)acrylic acid to form a reaction mixture.

This invention relates to a method for preparation of phenolic (meth)acrylates.

Phenolic (meth)acrylates typically are prepared using (meth)acrylic anhydride and a catalyst, e.g., in US2008227942. However, (meth)acrylic anhydrides are expensive and produce a mole of (meth)acrylic acid as a byproduct.

The problem solved by this invention is the need for improved preparations of phenolic (meth)acrylates.

### STATEMENT OF THE INVENTION

The present invention provides a method for preparation of phenolic (meth)acrylates; said method comprising contacting acetic anhydride, a phenolic compound and (meth)acrylic acid.

### DETAILED DESCRIPTION

Percentages are weight percentages (wt. %) and temperatures are in ° C. unless specified otherwise. Operations were performed at room temperature (20-25° C.), unless specified otherwise. The term “(meth)acrylic” means methacrylic or acrylic and “(meth)acrylate” means methacrylate or acrylate. Alkyl groups are saturated hydrocarbyl groups that may be straight or branched. Phenolic compounds include phenol, naphthols and substituted phenols. Non-hydrogen substituents on substituted phenols may be C₁-C₁₈ alkyl, alkenyl or alkynyl; halo (preferably bromo or chloro); hydroxyl and C₁-C₄ alkoxy. Preferably, non-hydrogen substituents on phenols are limited to C₁-C₄ alkyl, C₁-C₄ alkoxy and bromo or chloro; preferably methyl or methoxy. The hydroxyl group of phenol is not considered a substituent. Preferably, a substituted phenol has no more than three non-hydrogen substituents, preferably no more than two, preferably no more than one. A “solvent” is an organic compound which is a liquid at 20° C.

Typically, esterification reactions are performed in the presence of a strong acid catalyst, such as a heterogeneous acid catalyst (typically in the form of beads, especially an acidic polymer resin, e.g., sulfonic acid ion exchange resin beads), an organic sulfonic acid (e.g. methanesulfonic acid, benzenesulfonic acid, and toluenesulfonic acid), or a liquid acid such as sulfuric acid. Preferably, the reaction mixture in the present invention comprises no more than 0.5 wt % of acid catalysts, preferably no more than 0.1 wt %; preferably the reaction mixture is substantially free of heterogeneous catalysts, i.e., it contains no more than trace amounts of insoluble substances, i.e., amounts which may be present as impurities.

Preferably, the molar ratio of acetic anhydride to methacrylic acid is from 1:1 to 1:3; preferably no more than 1:2.5. Preferably, the molar ratio of acetic anhydride to phenolic compound is from 0.7:1 to 1:2; preferably at least 0.9:1; preferably no more than 1:1.1;.

Preferably, the reaction temperature is from 20 to 140° C.; preferably at least 35° C., preferably at least 40° C.; preferably no greater than 120° C., preferably no greater than 90° C.

Reaction times are dependent on temperature, but may easily be determined by those skilled in the art. Typically, reaction times are from 1 to 24 hours. Preferably, the acetic acid resulting from transesterification is removed by distillation after the reaction period at a temperature from 50 to 150° C. Preferably, the pressure in the reaction vessel is reduced from atmospheric pressure to a pressure from 1 to 200 mm Hg (1 to 26 kPa) to facilitate removal of acetic acid.

Preferably, (meth)acrylic acid, acetic anhydride and the phenolic compound are combined in a reaction vessel, preferably in the substantial absence of other components which are not solvents, i.e., the reaction mixture contains less than 2 wt. % of other components which are not solvents, preferably less than 1 wt. %, preferably less than 0.5 wt.

%, preferably less than 0.1 wt. %. All weight percentages of components in the reaction mixture are based on the total weight of the initial reactants (reaction mixture). Preferably, the reaction mixture contains less than the above limits of solvents. Preferably, the initial reaction mixture contains (meth)acrylic acid, acetic anhydride and the phenolic compound, with less than 2 wt. % of any other compounds other than (meth)acrylic acid and acetic acid, preferably less than 1 wt. %, preferably less than 0.5 wt. %. (Meth)acrylic acid and acetic acid are present as impurities in the respective anhydrides.

In one embodiment of the invention, sulfuric acid, strong organic acid, or strong acid resin is added to the reaction mixture, preferably in an amount no greater than 2 wt. %, preferably no greater than 1.5 wt. %, preferably no greater than 1 wt. %. Sulfuric acid may be added initially, during the initial reaction period or during the distillation of acetic acid.

### EXAMPLE

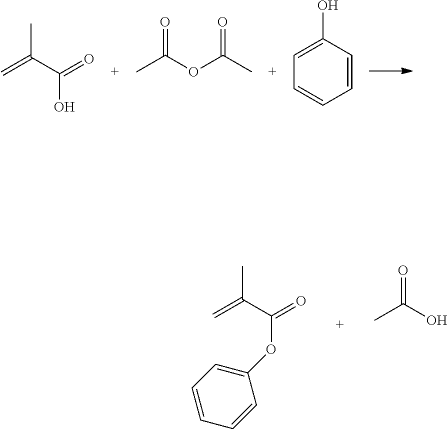

### Reactants

| | molecular | | | ratio to |
|---|---|---|---|---|
| | weight | mass, g | moles | phenol |
| methacrylic acid | 86.09 | 94.10 | 1.093 | 2.07 |
| acetic anhydride | 102.09 | 54.00 | 0.529 | 1.002 |
| phenol | 94.11 | 49.70 | 0.528 | 1.000 |

A 250 mL, 4-necked round bottom flask was fitted with a stir bar, a temperature controller, an over temperature monitor, a sampling/addition port, and a 10-plate Oldershaw distillation column with an attached automatic distillation head/reflux controller. The flask was charged with 94.1 g of glacial methacrylic acid, 49.4 g of phenol, 0.40 g PTZ inhibitor (2000 ppm concentration), and 54.0 g acetic anhydride. The contents of the flask were stirred for 6 hours at 50° C. NMR analysis of the pot contents after heating for 5 hours at 50° C. indicated that a fraction of acetic anhydride reacted with methacrylic acid to form methacrylic anhydride and a mixed anhydride. Some acetic acid was generated as a product. Unreacted phenol and methacrylic acid are still present in the contents of the flask.

The pressure of the flask was then reduced to 100 mm Hg, and the contents were heated to 83-85° C. Very little reflux was noted, so the pressure was reduced incrementally to 10 mm Hg. At this point, reflux initiated and the vapor temperature decreased from 56 to 35° C. as the pressure was reduced. About 5 mL of distillate was collected, and the distillation was halted. NMR analysis of the initial distillate taken indicated that it was composed almost entirely of acetic acid. Analysis of the pot contents after the first distillation indicate that a majority of the phenol starting material remained unreacted. No phenyl methacrylate was formed, and a limited amount of phenyl acetate and methacrylic anhydride were produced.

Further heating was resumed. The contents of the flask were heated at 84° C. under a pressure of 25 mm Hg over several hours. At a vapor temperature of 44° C., 35 mL of distillate was removed using a reflux ratio of 5:1. NMR analysis of the distillate revealed that the distillate was almost exclusively acetic acid. NMR analysis of the flask contents indicated that the majority of the product formed was phenyl acetate, and only a trace amount of phenyl methacrylate was produced. A significant amount of methacrylic anhydride were present in the reaction mixture, and a small amount of unreacted phenol was still present. The contents of the pot were heated again for an additional 4 hours at a pressure of 20 mm Hg. An additional 10 mL of distillate was collected at a vapor temperature range between 57 and 75° C. At this point, the pressure inside the flask was reduced to 15 mm Hg, and heating was continued for another 2.5 hours. Due to the lack of reflux, no additional distillate was collected in this step. Samples of the distillate and pot contents were drawn for NMR analysis. The distillate removed was comprised of a mixture of acetic acid and some methacrylic acid. Inside the reaction flask, unreacted phenol and methacrylic anhydride were still present in the reaction mixture, and a limited amount of the desired phenyl methacrylate was formed. The majority of the product composition in the flask was phenyl acetate (product ratio 29:71 phenyl methacrylate:phenyl acetate).

After cooling the pot contents to room temperature, 1 mL of concentrated H₂SO₄ was added to the flask. The solution color instantly changed from clear/colorless to red. The contents of the flask were then heated (pot temperature setting 110° C.) under a pressure of 20 mm Hg. Although a small amount of reflux was observed, no appreciable vapor reached the distillation head, and no additional distillate was collected during the reaction period. After 5 hours, the heating was discontinued and the flask restored to atmospheric pressure. A sample was drawn for NMR analysis. It was observed that the product composition of the mixture changed significantly. All of the phenol was consumed along with a significant majority of the methacrylic anhydride. A significant increase in the amount of phenyl methacrylate formed was observed. NMR analysis of the product mixture indicated that the product ratio in the reaction flask was now 65:35 phenyl methacrylate:phenyl acetate.

1. A method for preparation of phenolic (meth)acrylates; said method comprising contacting acetic anyhydride, a phenolic compound and (meth)acrylic acid to form a reaction mixture.

2. The method of claim 1 in which the reaction mixture contains less than 2 wt. % of other components which are not solvents.

3. The method of claim 2 in which the reaction mixture comprises no more than 0.1 wt. % of acidic catalysts.

4. The method of claim 3 in which the phenolic compound is phenol or phenol having non-hydrogen substituents which are selected from the group consisting of C₁-C₁₈ alkyl, alkenyl or alkynyl; bromo, chloro; hydroxy and C₁-C₄ alkoxy.

5. The method of claim 4 in which the reaction mixture is maintained in a temperature range from 20 to 100° C., followed by distillation of acetic acid at a temperature from 50 to 150° C. at a pressure less than normal atmospheric pressure.

6. The method of claim 5 in which the phenolic compound is phenol or a substituted phenol having no more than two non-hydrogen substituents.

7. The method of claim 6 in which the phenolic compound is phenol or a substituted phenol having non-hydrogen substituents selected from the group consisting of methyl, methoxy and hydroxy.

8. The method of claim 7 in which the reaction mixture comprises acetic anyhydride, a phenolic compound and methacrylic acid.

9. The method of claim 8 in which the phenolic compound is phenol.

10. The method of claim 3 in which the reaction mixture contains less than 1 wt. % of compounds other than (meth)acrylic acid, acetic anhydride and the phenolic compound.
